(19) United States
(12) Patent Application Publication
(10) Pub. No.: US 2008/0206864 A1
(43) Pub. Date: Aug. 28, 2008

(54) Methods and Materials for Culturing Murine Embryonic Stem Cells

(76) Inventors: Derrick Rancourt, Calgary (CA); Guoliang Meng, Calgary (CA)

Correspondence Address: FISH & RICHARDSON P.C., PO BOX 1022, MINNEAPOLIS, MN, 55440-1022, US

(21) Appl. No.: 11/943,312

(22) Filed: Nov. 20, 2007

Related U.S. Application Data
(60) Provisional application No. 60/866,991, filed on Nov. 22, 2006.

(51) Int. Cl. C12N 5/06

(52) U.S. Cl. 435/366; 435/377

(57) ABSTRACT

This disclosure provides methods for culturing murine ES cells in an undifferentiated state using human foreskin fibroblasts (HFF) feeder layer cells in the absence of exogenous Leukemia Inhibitory Factor (LIF).

### CROSS REFERENCE TO RELATED APPLICATIONS

This application claims priority under 35 U.S.C. §119 (e) of U.S. Application No. 60/866,991, filed Nov. 22, 2006.

### TECHNICAL FIELD

This invention relates to embryonic stem cells and, more particularly, to materials and methods for maintaining murine embryonic stem cells in an undifferentiated state.

### BACKGROUND

Embryonic stem (ES) cells are derived from the inner cell mass (ICM) of blastocysts. ES cells can be grown in culture and retain their full potential to produce all the cells of the mature animal, including its gametes. Mouse ESCs are routinely used to generate transgenic mice, as well as in cloning research. Furthermore they are routinely used as a model for cytotherapeutics in animal models. Traditionally, mouse ES cells have been cultured on mouse embryonic fibroblast (MEF) feeder layers in medium supplemented with mouse Leukemia Inhibitory Factor (LIF), which permits continuous growth of the ES cells in an undifferentiated state.

### SUMMARY

This disclosure describes novel and inexpensive methods for culturing murine ES cells in an undifferentiated state using feeder layer cells that include human foreskin fibroblasts (HFF). The methods described herein do not require exogenous Leukemia Inhibitory Factor (LIF).

In one aspect, the invention provides for methods of culturing murine embryonic stem (ES) cells in an undifferentiated state. Such a method generally includes culturing the murine ES cells on a feeder layer, thereby maintaining the murine ES cells in an undifferentiated state. The feeder layer typically includes human foreskin fibroblasts, and the culturing is performed in the absence of exogenous Leukemia Inhibitory Factor (LIF).

In one embodiment, the murine ES cells are mouse ES cells. Generally, the fibroblasts do not senesce for at least 60 passages. Typically, the culturing does not promote differentiation of the murine ES cells, does not reduce the expression of nucleic acid sequences associated with pluripotency of the murine ES cells, does not alter the ability of the murine cells to differentiate once removed from the feeder layer cells, and/or does not change the karyotype in the murine ES cells.

In another aspect, the invention provides for compositions that include murine ES cells, human foreskin fibroblasts and a culture medium that is substantially free of exogenous LIF (e.g., exogenous recombinant LIF). Such a composition can further include a culture substrate. Representative culture substrates include dishes, plates, flasks, bottles, or beads. In one embodiment, the murine ES cells are mouse ES cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 shows the results following the culture of three ES cell lines on human foreskin fibroblasts. After 20 passages on the human foreskin fibroblasts (HFF), ES cells were fixed and stained for alkaline phosphatase. Almost all colonies retained positive ALP staining. Additionally, ALP enzyme activity was measured in ES cell lines cultured on MEFs and after culture on HFFs.

FIG. 2 shows quantitative real-time expression analysis for pluripotency genes. Expression of cells cultured on HFFs for 20 passages was normalized to GAPDH and standardized to the respective starting passage cultured on MEFS, which was set as 1. No changes in gene expression were noted.

FIG. 3 shows CFC and EB assay and karyotype analysis. (A) ES cells cultured on HFF/−LIF retain the ability to form new colonies when seeded at clonal density. (B) They furthermore formed EBs in methylcellulose and when cultured in suspension. At day 8-9, a high percentage of the embryoid bodies had developed fluid filled cavities. (C) Karyotype analysis was performed on all three ES cell lines for two separate batches of each cell line after 10 and 20 passages of continuous culture on the human feeder layers, respectively. At least 50 metaphases from each cell line were examined in order to establish their chromosome number; each one was found to possess a normal 46, XY karyotype.

FIG. 4 shows cytokine expression by human foreskin fibroblasts. ELISA was used to determine human LIF activity in the cell culture supernatant after incubation of the foreskin fibroblasts with medium for 1 h, 2 h and 3 h.

Like reference symbols in the various drawings indicate like elements.

### DETAILED DESCRIPTION

This disclosure provides novel and inexpensive methods for culturing murine embryonic stem (ES) cells and maintaining such ES cells in an undifferentiated state. The methods include culturing murine ES cells on a feeder layer of human foreskin fibroblasts (HFF). Murine ES cells can be mouse ES cells or other ES cells from species within the Mus genus. Common murine ES cells and methods of obtaining and culturing ES cells are described, for example, in U.S. Pat. No. 5,166,065; Koestenbauer et al., 2006, Am. J. Reprod. Immunol., 55:169-80; Well, 2002, Methods Mol. Biol., 180:93-126; and Tessarollo, 2001, Methods Mol. Biol., 185:27-33. See, also, information provided by the ES Cell Core at Washington University, St. Louis, Mo. (on the World Wide Web at escore.im.wustl.edu).

HFF for use as feeder layer cells can be obtained commercially (e.g., PromoCell GmbH, Heidelberg, Germany) or can be prepared using standard methods (e.g., Vilcek et al., 1978, Adv. Exp. Med. Biol., 110:101-18; Weinstein et al., 1982, FEBS Lett., 144:85-8; or Example 1 below). When used as feeder layer cells, HFF are able to survive for more than 60 passages before senescence is observed.

Prior to the present invention, Leukemia Inhibitory Factor (LIF) was added to cultures of ES cells to keep the ES cells from differentiating. See, for example, U.S. Pat. No. 5,166,065. LIF is a cytokine of the IL-6 family and, although not bound by any particular mechanism, has been shown to regulate expression of the pluripotency gene Oct-4 through the JAK/STAT signaling pathway. LIF also has been shown to be sufficient to support long-term in vitro growth of undifferentiated murine ES cells. When human foreskin fibroblasts are used as feeder layer cells to culture murine ES cells, however, the culture medium does not require the addition of exogenous LIF. As used herein, exogenous LIF refers to LIF (e.g., recombinantly produced LIF) that is added to the culture and that is not produced by the feeder layer or ES cells.

Media used to culture murine ES cells is well described and can be obtained commercially (see, for example, Invitrogen (Carlsbad, Calif.) and Celprogen (San Pedro, Calif.). Culture medium generally includes a carbohydrate source (e.g., glucose and/or sodium pyruvate), serum or serum replacer, amino acids and inorganic salts. Dulbecco's Modified Eagle's Medium (DMEM) is a common medium used in cell culture and can be supplemented and/or modified for particular cell types as necessary. See, also, Tessarollo (2001, Methods Mol. Biol., 185:27-33) for a description of mouse ES cell culture methods.

It is understood by those in the art that culturing ES cells in the absence of differentiation is sometimes referred to as expansion. The culturing methods disclosed herein are directed toward methods of expanding ES cells and, therefore, do not cause a reduction in the expression of sequences associated with pluripotency (e.g., Oct4, nanog or APL) in the murine ES cells. In addition, the methods disclosed herein do not change the karyotype of the ES cells and, further, do not alter the ability of the ES cells to differentiate once removed from the feeder layer cells.

In addition to the methods described herein, a composition that includes murine ES cells, HFF, and culture medium substantially free of exogenous LIF also is described. As used herein, “substantially free of exogenous LIF” means that LIF (e.g., recombinantly produced LIF) is not added to the culture medium, although it is understood by those of skill in the art that “endogenous” LIF may be present in the culture medium if the feeder layer cells and/or the ES cells produce LIF.

As would be apparent to those of skill in the art, such a composition also can include any of a number of different culture substrates for supporting the growth of the cells. Culture substrates can include, without limitation, dishes, plates, flasks, bottles or beads.

The methods and compositions disclosed herein are particularly useful, for example, in high-throughput culturing of ES cells. Eliminating the need to add exogenous LIF significantly reduces the cost associated with culturing ES cells. In addition, the lengthy lifespan of the human foreskin fibroblasts reduces the labor involved in culturing ES cells and reduces the amount of batch-to-batch variation.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### Isolation and Culture of Human Foreskin Fibroblasts (HFF)

Human foreskins were obtained from the local clinic, washed in PBS containing penicillin/streptomycin and minced. Single cells were obtained by trypsinization for 30 min and seeded into culture medium (high-glucose DMEM containing 10% FBS, 2 mM L-glutamine, 0.1 mM 2-mercaptoethanol and 1% non essential amino acids). Cells were passaged every second day at a ratio of 1:3.

### Example 2

### Isolation of Murine Embryonic Fibroblasts (MEF)

Female mice of the CD1 strain were routinely bred at the University of Calgary animal facility. At day 12.5 of pregnancy, pregnant animals were sacrificed by cervical dislocation. Embryos were flushed from the uteri horns and killed by decapitation. Embryos were washed in PBS and the head and the placenta were removed. The carcasses were minced, soaked in trypsin at 4° C. overnight and dissociated in a final 5 min incubation step at 37° C. Tissue was broken up by repeated pipetting and larger remaining clumps removed by letting them settle out. Cells were seeded in DMEM supplemented with 10% fetal bovine serum and cultured for up to five passages.

### Example 3

### Preparation of Feeder Layers from MEF and HFF

When the MEF and HFF reached confluency, mitomycin C was added to the cells to a final concentration of 10 μg/ml. After incubation for 2 h and 3 h at 37° C., respectively, the medium was aspirated off and the cells were washed five times with PBS, then trypsinized, centrifuged, suspended, and re-plated at appropriate concentrations. The prepared feeder layers were used the following day up to one week.

### Example 4

### Embryonic Stem Cell Culture

R1 ES cells were obtained from Dr. Andras Nagy (University of Toronto). Bruce4 (B4) ES cells were obtained from Dr. Frank Koentgen (OZGENE, Australia). 9C12 was an ES cell lines derived from F1 (129×C57BL6) prior to these experiments by G. Meng.

In brief, 3.5-dpc blastocysts were flushed from the uterine horns with DMEM plus 10% serum and placed individually into 10 mm well tissue culture dishes containing a preformed MEF feeder layer and 0.5˜0.6 ml ES cell medium (1000 U/ml MLIF, Life Sciences). One to two days later, the embryos hatched from the zona pellucida and attached to the surface of the tissue culture dish with spreading of the trophoblast cells. Four to five days after explantation into culture, the outgrowth was dislodged from the underlying sheet of trophoblast cells using a finely drawn pasteur pipette. The outgrowth was washed with PBS and transferred to a microdrop of 0.25% trypsin/0.2% EDTA in PBS. The ICM clump was gently disaggregated with a pipette into smaller cellular aggregates of two or three cells, which were transferred into a fresh 10 mm feeder cell tissue culture well. Generally, after 2 days, primary colonies of cells would become readily visible. 9C12 is characterized by high competent transmission to the germline identified by microinjection and aggregation.

All three ES cell lines were routinely grown on murine feeder layers isolated as described above in DMEM (4.5 g glucose/l) containing 20% FBS, 2 mM L-glutamine, 0.1 mM 2-mercaptoethanol and 1% non essential amino acids with the addition of Leukemia Inhibitory Factor (1000 U/ml, Life Sciences). ES cell lines were fed daily and passaged every two days. For subsequent analysis, ES cell cultures were dissociated with 0.25% Trypsin/EDTA and pre-plated into tissue culture dishes for 40 min. ES cells in the supernatant were harvested leaving the feeder cells behind. For transfer of ES cell lines from MEF onto HFF, a similar pre-plating technique was utilized. After removal of MEF feeder cells, each mES cell line was transferred to the foreskin feeders (in ES cell medium with and without LIF, respectively). Each transferred line was continuously cultured for an additional 20 generations at a splitting ration of 1:5.

### Example 5

### ALP Stain and Enzyme Activity

Cultures were thoroughly rinsed twice in PBS and lysed in RIPA buffer (150 mM NaCl, 10 mM Tris, pH 7.2, 0.1% SDS, 1% Triton X-100, 1% Deoxycholate, 5 mM EDTA) containing 1 mM phenylmethylsulfonyl fluoride, 10 mM benzamidine, 2 μg/ml leupeptin, 100 μM sodium orthovanadate and 10 mM p-nitrophenylphosphate. After gentle rocking for 30 min, lysates were collected, centrifuged and an aliquot of the supernatant was subjected to subsequent analysis. Alkaline phosphatase activity was determined from the protein lysates using p-nitrophenyl phosphate (Sigma) as a substrate. The production of yellow p-nitrophenol was stopped with 3 N NaOH after a 20 min incubation at 37° C. Absorbance was read at t=0 and t=20 min at 405 nm. Enzyme activity was calculated as previously described (zur Nieden et al., 2003, Differentiation, 71:18-24). ALP enzyme activity of all cultures was normalized to the total protein content of the samples. Protein content of the lysates was determined after the Lowry method with the DC protein kit from BioRad as previously described (Cormier et al., 2006, Tissue Eng., e-pub October 1).

### Example 6

### Immunocytochemistry and Flow Cytometry

ES cells were fixed with ice-cold methanol:acetone and permeabilized with PBS/0.1% Triton-X 100. Unspecific binding was blocked with PBS/1% BSA for 30 min at room temperature. Cells were overlaid with an anti-Oct4 antibody (Chemicon), incubated at 4° C. overnight and visualized with an Alexa Fluor 488 conjugated secondary antibody (Molecular Probes, Temecula, USA).

For flow cytometry, suspensions of ES cells were washed in PBS. Cell numbers and viabilities were determined using hemocytometer counts with trypan blue exclusion. Two million cells per sample group were fixed in 2% PFA and prepared for FACS analysis as described (Cormier et al., supra). Briefly, for the detection of intracellular antigens, blocking buffer contained 0.15% Saponin. Ten thousand events were registered per sample and analysis was performed using appropriate scatter gates to avoid cellular debris and aggregates using a FACS Calibur instrument and the CellQuest software from Becton Dickinson. A cell was considered to be positively stained if the measured fluorescence intensity exceeded the signal obtained from cells incubated with secondary antibody only.

### Example 7

### RT-PCR and Quantitative PCR

Cells were harvested for RNA isolation in triplicate, which was performed using the RNeasy Midi Kit from Qiagen according to the manufacturer's instructions. Genomic DNA was digested with DNase I on column. RNA concentration was determined with the Ribogreen RNA quantitation reagent and kit (Molecular Probes) as previously described (zur Nieden et al., 2001, Toxicology in Vitro, 15:455-461). Complementary DNA was synthesized from 500 ng of RNA with Superscript II (Invitrogen) as previously described (zur Nieden et al., 2005, Dev. Biol., 5:1-5). One tenth of the cDNA reaction was then subjected to PCR. Reactions further contained 0.8 μM of each of the primers, 2 mM MgCl₂, 0.1 mM of each of the dNTPs and 2.5 U of Taq Polymerase in 1×PCR buffer (Invitrogen) in a total reaction volume of 25 μl and were cycled through 35 rounds of 30 s at 94° C., 30 s at the corresponding annealing temperature and 30 s at 72° C. Primer sequences for amplification of human genes were as follows: hLIF, forward 5′-TGA ACC AGA TCA GGA GCC AAC T-3′ (SEQ ID NO:1), reverse 5′-CCA CAT AGC TTG TCC AGG TTG TT-3′ (SEQ ID NO:2); hb-FGF, forward 5′-CCG TTA CCT GGC TAT GAA GG-3′ (SEQ ID NO:3), reverse 5′-ACT GCC CAG TTC GTT TCA GT-3′ (SEQ ID NO:4); hGAPDH, forward 5′-CAA CGG ATT TGG TCG TAT TGG-3′ (SEQ ID NO:5), reverse 5′-GCA ACA ATA TCC ACT TTA CCA GAG TTA A-3′ (SEQ ID NO:6) and primer sequences for murine genes were described previously (Cormier et al., supra). DNA products were fractionated on 3% agarose gels at 70V for 90 min. Quantitative PCR was carried out as described (zur Nieden et al., 2005, supra) and expression of genes of interest was normalized to GAPDH.

### Example 8

### CFC and EB Assay

ES cells were tested for their capability to reform new colonies and to form embryoid bodies in methylcellulose as described previously (Cormier et al., supra). Emerging colonies and embryoid bodies from n=3 dishes were counted after 5 days.

### Example 9

### Embryoid Body Formation

ES cell colonies were passaged at normal density onto gelatinized plates to free the culture of contamination by fibroblast cells. For embryoid body formation, the cells were trypsinized as for normal passaging until the colonies lifted off. Loosely connected clumps of cells were kept together by gentle handling. Cells were directly plated in a 1:3 ration into petri dishes coated with a layer of agar in LIF-free ES cell medium. Medium was replaced every two days.

### Example 10

### Karyotyping

ES cell cultures were treated with 0.1 μg/ml colcemid (Invitrogen) for 1 hour to induce metaphase arrest. The colonies were then dissociated with trypsin, the cells incubated in 0.075 M KCl for 20 min and fixed in glacial acetic acid:methanol (1:3). Cells were spread onto a slide and chromosomes counted for n=50 cells.

### Example 11

### Teratoma Formation and Histological Analysis

C57 BL/6 mice were obtained from Charles River and housed in the single-barrier animal facility of the Faculty of Medicine, University of Calgary. About 2×10⁷ cells were injected into the skin fold of the inner thigh of four mice per group. Animal protocols were carried out as approved by the University of Calgary (Animal Protocol # 157-GM). Animals were sacrificed after 4 weeks. Teratomas were dissected out, fixed in formalin over night and embedded in paraffin. Sections were stained in hematoxylin/eosin according to standard procedures (Loken et al., 2004, Cancer Biol. Ther., 3:734-738).

### Example 12

### Aggregation of ES Cells and Eight Cell-Stage Embryos

Oviducts with the upper part of the uterus attached were removed from 2.5 days post-coitum (dpc) superovulated CD-1 females and placed into a drop of M2 (Sigma). Under dissecting microscope, the oviducts were flushed by inserting the flushing needle attached to a 1 ml syringe of M2 into the infundibulum. 8-cell stage embryos were collected using mouth controlled pipette and washed through several drops of M2 medium to remove any debris. The embryos were washed in KSOM medium and cultured in organ culture dish in KSOM at 37° C. with 5% CO₂. The zona pellucida was removed from the embryo by incubation in M2, KSOM containing acid Tyrode's. Embryos were washed at least twice in KSOM drops after dissociation was complete before putting them in an aggregation plate. The embryo was then placed on top of a clump of loosely connected ES cells and cultured over night at 37° C., 5% CO₂. A maximum of 8-10 embryos were then transferred into each uterine horn of a 2.5 dpc pseudopregnant recipient. Mature CD-1 females were used as pseudopregnant foster mothers and ordered at a weight of >30 g. Chimerism of newborn mice was assessed by coat color inspection, where the coat color derived from hybrid ES cell was agouti, while that from the host embryo was white. Six weeks after birth, chimeric mice with high ES cell contribution (>75% agouti coat color) were crossed with CD1 mice and the germ-line transmission was judged by the presence of agouti pups.

### Example 13

### Enzyme-Linked Immunosorbent Assay (ELISA)

Concentrations of secreted human LIF were determined with the human LIF Quantikine ELISA kit from R&D Systems according to the manufacturer's instruction. HFF were grown to confluency and medium samples were taken 1 h, 2 h, and 3 h after the medium was changed. Cells were then lysed in HCl [0.1 M] to determine LIF secretion per amount of protein.

### Example 14

### Statistical Analysis

Differences in gene expression, enzyme activities and other analysis between P0 and P20 ES cell cultures were evaluated with an unpaired student's t-test (physics.csbsju.edu/stats/t-test.html on the World Wide Web). None of the differences were found to be statistically significant.

### Example 15

### Murine ES Cells Can be Expanded on Human Foreskin Fibroblasts without LIF

Human foreskin fibroblasts were cultured for 62 passages without overt senescence. Feeder layers prepared from the human foreskin fibroblasts organized directionally in a manner typical for fibroblasts.

The three ES cell lines (R1, Bruce4 and 9C12) were at P6, P19 and P21 when they were transferred onto human fibroblast feeders. These passage numbers were designated P0 for this experiment and the cells were passaged onto human fibroblasts for an additional 20 passages (P20). Human foreskin fibroblasts used to maintain mouse ES cells were at passage 25-30. After 20 passages cultured in foreskin feeders in ES cell media without LIF (HFF/−LIF), the morphology of ES cell colonies that grew on the human feeder layers were slightly different from the ones cultured on MEF with LIF (MEF/+LIF). It seemed that the cells were organized according to the direction of the foreskin feeder layers. Consequently, the colonies are not as round as those grown on MEF. However, the individual mouse ES cell morphology remained the same as that of cells cultured on MEF with LIF. The cells remained round and small, with a high nucleus to cytoplasm ratio and a presence of one to three nucleoli.

### Example 16

### Stem Cell Marker Expression

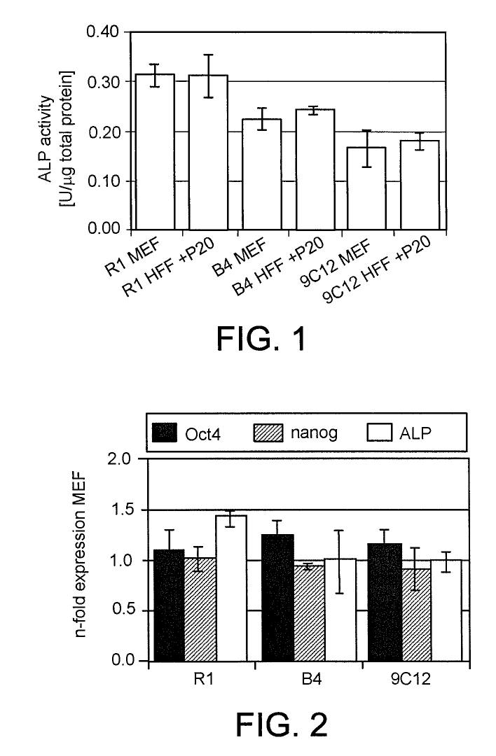

In common with pluripotent stem cells, the three ES cell lines (R1, Bruce4 and 9C12) showed high levels of alkaline phosphatase activity (FIG. 1). Comparable levels were found for corresponding P0 (MEF/+LIF) and P20 (HFF/−LIF) cultures. However, although all three ES cell lines were found to be pluripotent in subsequent analyses, it should be noted that ALP activity varied between the three ES cell lines intensively.

Positive expression of Oct-4 was observed by immunocytochemistry and flow cytometry in all three lines after 20 passages on human fibroblast feeders. All three ES cell lines were over 95% positive for Oct-4, a quality control marker for ES cells. In fact, R1 ES cells showed a percentage of 99.91% cells, B4 cultures contained 99.47% and 9C12 ES cell cultures 97.03% Oct-4 positive cells after 20 passages on the human feeders.

Expression of the pluripotency marker genes Oct-4, nanog and rex-1 remained comparable in all ES cell lines after culture on HFF for 20 passages as judged by RT-PCR. Solely, ALP expression seemed to decrease in B4 ES cells over time when cultured on HFF/−LIF, but subsequent expression analysis by quantitative PCR confirmed that expression levels were not altered (FIG. 2). Brachyury message was barely detectable in P0 cultures and slightly up-regulated with continuous culture on HFF, whereas neither AFP mRNA as a marker for endodermal differentiation nor NF68 mRNA expression (ectoderm) could be detected. Expression of the recently described general differentiation marker 5T4, was detectable in all cultures, which likely originated from contamination with feeder cells or from differentiating ES cells. Although ES cells were pre-plated for 40 min, which should remove most of the feeder cells, carry-over might have been possible. Since only brachyury as an early differentiation marker was expressed by the ES cells in very low levels, the expression of 5T4 was ascribed to contamination with feeders.

### Example 17

### ES Cells are Capable of Forming Novel Colonies and Embryoid Bodies

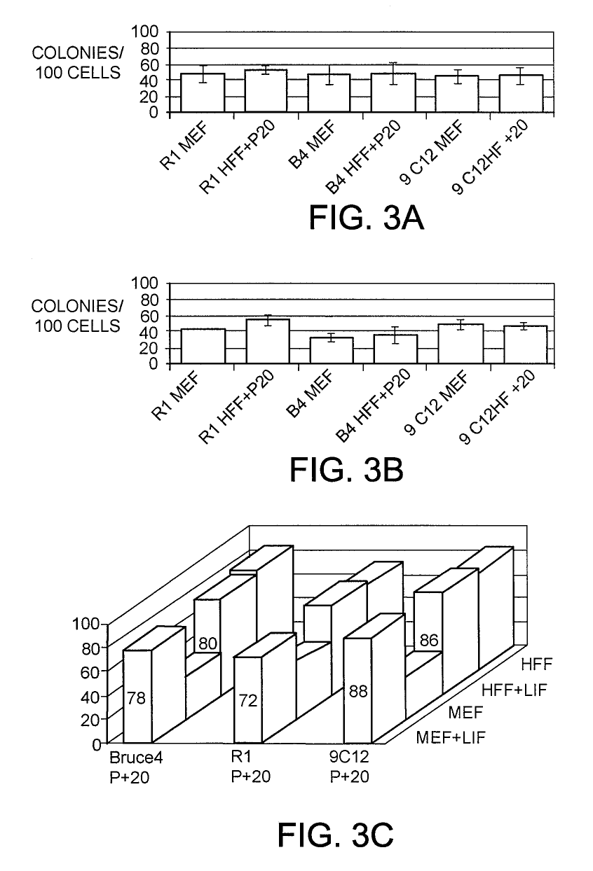

To assess the level of pluripotency of the three ES cell lines cultured on human foreskin fibroblasts for 20 passages, colony forming efficiencies and EB formation assays were compared between cells from P0 (MEF/+LIF) and P20 (HFF/−LIF). It was found that all three ES cell lines retained the ability to form colonies at a comparable efficiency of approximately 45-55% (FIG. 3A). With respect to the EB formation assays, cells from all cultures formed EBs at comparable levels (FIG. 3B). With EB formation efficiencies of 38-54 per 1000 cells, similar values were found for all three cell lines as previously reported [Cormier et al., supra]. From day 9 to day 10, a high percentage of the embryoid bodies had developed fluid filled cavities, described as cystic embryoid bodies. As these represent cell types of all three germ layers, they only develop from pluripotent ES cells, thus providing support to the notion that the ES cells are truly pluripotent after long-term culture on HFF/−LIF.

Karyotype analysis was performed at 20 passages and all three ES cell lines retained a normal karyotype (46-XY) after culture on MEF and human foreskin fibroblasts with LIF (FIG. 3C). MEF without additional LIF supplementation (MEF/−LIF) were not able to sustain a normal karyotype over time. However, HFF even without exogenous addition of LIF supported the maintenance of a normal ES cell karyotype.

### Example 18

### ES Cells Form Teratomas Containing all Three Germ Layers

The ES cell line, Bruce4, cultured for 20 passages on HFF in LIF-free medium was injected into severe combined immunodefiency (SCID) mice to confirm the formation of teratomas. Bruce4 ES cells (HFF/−LIF) formed teratomas in each injected mouse and they contained representative tissues of all three germ layers, such as nervous fiber, cartilage, blood vessel, skeletal muscle, stratified cuboidal epithelium, and simple cuboidal ciliated epithelium, etc.

### Example 19

### ES Cells Cultured on HFF without LIF are Capable of Contributing to the Germline

After culture in LIF-free DMEM ES medium on foreskin feeder layers for six passages, hybrid ES cells were aggregated with diploid eight-cell stage embryos flushed from CD1 pregnant mice. When these aggregates developed into morula or blastocyst stage, they were transferred into CD1 pseudo female mice. The aggregation method for generating chimaeras, as opposed to the microinjection technique, is useful as it does not require expensive microinjection apparatus or sophisticated manipulative skills and utilizes outbred blastocysts, which are easier to obtain in numbers. The ES cells were introduced into the developing embryo by the adherence of the cells to a dezonulated eight-cell stage embryo. The embryos were cultured to blastocysts and then transferred into a pseudopregnant CD1 mouse. Chimerism of newborn mice was assessed by coat color inspection, where the coat color derived from hybrid ES cell was agouti while that from the host embryo was white. Six weeks after birth, chimeric mice with high ES cell contribution (>50% agouti coat color) were crossed with CD1 mice and the germ-line transmission was judged by the presence of agouti pups.

### Example 20

### HFF Express Cytokines that Support ES Cell Pluripotency

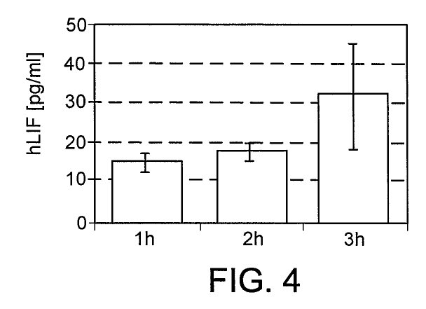

Since HFF were capable of supporting ES cell pluripotency without exogenous addition of recombinant LIF, we were interested to see whether HFF expressed LIF themselves. Moreover, since HFF are also used for maintenance of undifferentiated human ES cells, an expression analysis for b-FGF was performed. Both mRNAs were expressed by early (P6) as well as late passage (P32) HFF. The corresponding LIF protein is secreted into the medium by the fibroblasts as was determined by ELISA for the human LIF protein (FIG. 4). LIF was secreted at an average rate of 11.47±5.5 pg/ml per hour, which corresponds to 2.32±0.62 fg per ng total cellular protein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of culturing murine embryonic stem (ES) cells in an undifferentiated state, comprising:
culturing said murine ES cells on a feeder layer, wherein said feeder layer comprises human foreskin fibroblasts, wherein said culturing is in the absence of exogenous Leukemia Inhibitory Factor (LIF),
thereby maintaining said murine ES cells in an undifferentiated state.

2. The method of claim 1, wherein said murine ES cells are mouse ES cells.

3. The method of claim 1, wherein said fibroblasts do not senesce for at least 60 passages.

4. The method of claim 1, wherein said culturing does not promote differentiation of said murine ES cells.

5. The method of claim 1, wherein said culturing does not reduce the expression of nucleic acid sequences associated with pluripotency of said murine ES cells.

6. The method of claim 1, wherein said culturing does not alter the ability of said murine cells to differentiate once removed from said feeder layer cells.

7. The method of claim 1, wherein said culturing does not change the karyotype in said murine ES cells.

8. A composition comprising murine ES cells, human foreskin fibroblasts and a culture medium substantially free of exogenous LIF.

9. The composition of claim 8, further comprising a culture substrate.

10. The composition of claim 9, wherein said culture substrate is selected from the group consisting of a dish, a plate, a flask, a bottle, or a bead.

11. The composition of claim 8, wherein said murine ES cells are mouse ES cells.

12. The composition of claim 8, wherein said culture medium is substantially free of exogenous recombinant LIF.
